(19) United States
(12) Patent Application Publication
(10) Pub. No.: US 2009/0246166 A1
(43) Pub. Date: Oct. 1, 2009

(54) Pearl Like Polyvinyl Alcohol Particles, Production Process Thereof and Temporary Vascular Embolic Agent

(75) Inventors: Mitsuo Shibutani, Osaka-shi (JP); Kazutoshi Tsuji, Osaka-shi (JP)

Correspondence Address: Rahman LLC, 10025 Governor Warfield Parkway, Suite 110, Columbia, MD, 21044, US

(73) Assignee: The Nippon Synthetic Chemical Industry CO., LTD., Osaka-shi (JP)

(21) Appl. No.: 12/410,749

(22) Filed: Mar. 25, 2009

(30) Foreign Application Priority Data
Mar. 25, 2008 (JP) 2008-78929

(51) Int. Cl. A61K 31/765; A61P 9/14; C08F 16/06; C08F 8/12

(52) U.S. Cl. 424/78.18; 525/56; 525/62

(57) ABSTRACT

The embodiments provide a temporary vascular embolic agent temporarily blocking the blood vessel in vivo, having appropriate dissolution speed suitable for use in the blood vessel in which blood flow is fast and being more superior in the passing property of a catheter and the controllability of embolization time than conventional one. The embolization of the blood vessel led to cardiac muscle in which blood flow speed is fast is enabled by the temporary vascular embolic agent of the present invention using the pearl like polyvinyl alcohol (PVA) particles having 0.1 to 2% by mol of a 1,2-diol configuration unit and having a saponification degree of at least 99% by mol.

### CROSS-REFERENCE TO RELATED APPLICATIONS

This application claims the benefit of Japanese Patent Application No. 2008-78929 filed on Mar. 25, 2008, the complete contents of which, in its entirety, is herein incorporated by reference.

### BACKGROUND OF THE INVENTION

The present invention relates to pearl like polyvinyl alcohol particles involving a vascular embolic agent temporarily blocking blood vessels and used for temporarily blocking blood flow in vivo, and specifically used for an embolic material.

There has been known artery embolotherapy blocking nutrition by vascular blockage for tumor and fibroid unable to be cut in advance of incision accompanied by surgical operation, except purpose for minimizing bleeding and purpose for preventing bleeding. Further, there has been known chemical embolotherapy blocking blood flow in a tumor by dosing in combination of an anticancer drug with a vascular embolic agent and highly keeping the concentration of the anticancer drug to desire the improvement of an anticancer effect. As these embolic agents injected into the blood vessels, a dimethyl sulfoxide solution (hereinafter, called as DMSO) of about 10% by weight of EVOH, cyanoacrylate and formal particles of polyvinyl alcohol (hereinafter, called as PVA) have been known.

Among these embolic agents, for example, EVOH (about 10% by weight of DMSO solution) affects badly living organism from the viewpoint that DMSO being a solvent has toxicity. Cyanoacrylate can control embolic time in the blood by controlling a mixing ratio with the saponified article of iodoaliphatic ester but balance with the mixing ratio is difficult. Accordingly, when timing of drawing a catheter after dosing in the blood vessel through the catheter is mistaken, the edge of the catheter adhering in the blood vessel and there is a risk that the edge of the catheter remains in the blood vessel in worst case. Further, the formal particles of EVOH and PVA are permanent embolic agents and there has been a problem that they cannot be used for use in temporary embolization.

Various studies have been carried out for solving these problems. For example, in the patent literature, although a gelatin sponge is used for temporary embolization, there is a risk that a gelatin specifically obtained from cattle as a raw material mediates infection such as mad cow disease because the gelatin sponge includes components derived from an organism. Furthermore, when it is used as a temporary embolic material, it is necessary to very finely cut it at a thickness of about 1 mm before use in order to pass the gelatin sponge through the catheter. The very high skillfulness of a doctor is required and there is a problem that differences in individuals occur in a remedy effect. Further, although crosslinked starch is also used as the temporary embolic material in general, it is decomposed in minutes by amylase in blood, therefore it has not been an embolic agent effective for a comparatively long fixed period for one week to 3 months.

The patent literature 2 discloses a vascular embolic agent having water swelling rate of at least 30% and comprising granular particles degraded in phosphoric acid buffer saline. However, the vascular embolic agent was obtained by the block copolymerization of a water soluble polymer by addition of a biodegradable component or by crosslinking or modification, and its decomposition is due to biodegradation; therefore the control of embolization time in the blood vessel has been inadequate yet.

The patent literature 3 discloses a temporary vascular embolic agent using pearl like polyvinyl alcohol particles. The temporary vascular embolic agent is absorbed in vivo and then, evacuated naturally in vitro. Further, it has no risk of the propagation of infections such as mad cow disease and AIDS mediating the blood and further, there is a small risk of provoking coagulation blockage in the blood vessel for purposes other than original intent. Accordingly, it does not provoke stack and it is indicated that passing property in a catheter is good. When the temporary vascular embolic agent is used for blocking nutrition and for blocking blood flow in a tumor and highly keeping the concentration of an anticancer drug, it is adequate to control dissolution speed by a saponification degree because the precise control of embolization time is unnecessary.

However, the cardiac infarction model using animals is required for the development of the therapeutic agent and treatment procedure of cardiac infarction and the evaluation of pharmacological effect. In conventional cardiac infarction models, a method of embedding tools in which a water-absorbing resin is a composing component, in the blood vessel led to cardiac muscle by thoracotomy and a method of carrying out the infarction of blood vessel by surgical treatment such as direct binding are carried out. However, the surgical methods provide great load to animals, success rate is often low at 50% because of operational death, there is also limit to an applicable blood vessel size, the preparation of infarction generated at capillary blood vessel sites is difficult and the reproducibility of the range and level of infarction is also low; therefore the methods have not been said to be an adequate good preparation method of a cardiac infarction model.

In order to prepare the cardiac infarction model, a temporary vascular embolic agent is injected in the nutritious artery of cardiac muscle using a catheter, to carry out the infarction of the blood vessel, a cardiac infarction state is prepared by stopping blood flow to cardiac muscle, and then, it is adequate that normal blood flow can be restored after the lapse of specific time.

However, in the case of the preparation of the cardiac infarction model, it is necessary to block the artery and a temporary vascular embolic agent having not too fast dissolution speed and having appropriate dissolution speed is required because the dissolution speed is apt to be great in the artery in which blood flow rate is fast.

Further, it is required to heighten the reproducibility of embolization time in order to prepare cardiac infarction state in which the level and range of infarction is constant, in good reproducibility; therefore the more precise control of dissolution speed than conventional one has been required.

The contents of the entireties of the following patent publications are incorporated by reference herein: International Patent Publication WO98/03203, Japanese Unexamined Patent Publication No. 2004-167229, and Japanese Unexamined Patent Publication No. 2007-37989.

### SUMMARY OF THE INVENTION

It is an object of the present invention to provide a temporary vascular embolic agent having appropriate dissolution speed suitable for use in the blood vessel in which blood flow is fast and being more superior in the passing property of a catheter and the controllability of embolization time than conventional one.

Namely, the present invention relates to a pearl like polyvinyl alcohol particle including a polyvinyl alcohol resin having 0.1 to 2% by mol of a 1,2-diol configuration unit indicated by the general formula (1) and having a saponification degree of at least 99% by mol.

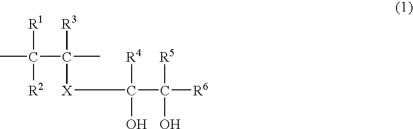

[Wherein R¹, R² and R³ independently indicate a hydrogen atom or an organic group respectively, X indicates a single bond or a bond chain, and R⁴, R⁵ and R⁶ independently indicate a hydrogen atom or an organic group respectively.]

Further, the present invention relates to a process for producing the pearl like polyvinyl alcohol particle of claim 1, wherein a polyvinyl ester copolymer solution obtained by copolymerizing a vinyl ester monomer with a monomer capable of being the 1,2-diol configuration unit indicated by the general formula (1) by saponification is saponified while being dispersed in a specific high viscous liquid.

R¹, R2, R3, R4, R5 and R6 in the general formula (1) are preferably hydrogen atoms or alkyl groups having 1 to 4 carbon atoms.

Further, as another aspect of the present invention, R¹, R², R³, R⁴, R⁵ and R⁶ in the general formula (1) are hydrogen atoms and X is a single bond.

Further, the present invention relates to a temporary vascular embolic agent including the pearl like polyvinyl alcohol particle.

The embolic agent of the present invention is naturally evacuated in vitro after absorption in vivo. Further, since it is not a temporary embolic agent derived from the blood, there is no risk of propagating infections such as mad cow disease and AIDS mediating the blood and further, there is a small risk of provoking coagulation blockage in the blood vessel for purposes other than the intended vessel. Consequently, according to the present invention, it does not provoke stack and passing property in a catheter is good. Further, since the crystallization of polyvinyl alcohol is easily controlled, it exhibits suitable dissolution speed even in the case of the blood vessel with fast blood flow speed such as the nutrition artery of cardiac muscle and further, the pearl like polyvinyl alcohol particles for a temporary vascular embolic agent capable of precisely controlling embolization time and the embolic agent can be provided.

### DETAILED DESCRIPTION

The particles of the present invention are the pearl like polyvinyl alcohol particles having 0.1 to 2% by mol of a 1,2-diol configuration unit and having a saponification degree of at least 99% by mol. Solubility for water can be precisely controlled by the content of such a configuration unit or thermal processing condition. Herein, the pear like particle is not a granular or heteromorphous powdery article but represents particles having a constant sphericity.

The sphericity is prescribed by sphericity coefficient. Herein, the scanning electron micrograph of powders is photographed, the length (L) of contours concerning individual particles observed in the unit field of the photo and the circumference (M) of a circle having the same area as the area of particles are measured, and the average value of values (M/L) obtained by dividing M with L is prescribed as sphericity coefficient. In general, when the sphericity coefficient is at most 0.96, the effect for embolizing the blood vessel is lowered, therefore the sphericity coefficient of the pearl like polyvinyl alcohol (PVA) particles is preferably at least 0.96. The sphericity coefficient is preferably at least 0.98 and further preferably at least 0.99.

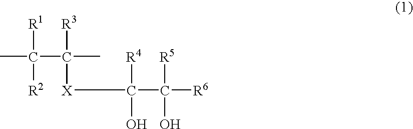

[Wherein R¹, R² and R³ independently indicate a hydrogen atom or an organic group respectively, X indicates a single bond or a bond chain, and R⁴, R⁵ and R⁶ independently indicate a hydrogen atom or an organic group respectively.]

The pearl like polyvinyl alcohol particles of the present invention have 0.1 to 2% by mol of a 1,2-diol configuration unit indicated by the general formula (1) in its molecule and so far as the particles have a small amount of the specific 1,2-diol configuration unit, they are particularly suitable for a temporary vascular embolic agent for preparing a cardiac infarction model. Here, 0.2 to 1% by mol is more preferable and 0.3 to 0.5% by mol is further preferable. When the introduction quantity of the configuration unit exceeds 2% by mol, embolization time is not preferably short because dissolution speed is too fast. On the other hand, when the introduction quantity of the configuration unit is less than 0.1% mol, the effect of modification is small, the fine adjustment of crystallinity is difficult and the control of dissolution speed in blood is difficult.

Herein, the content rate of the 1,2-diol configuration unit included in a PVA resin can be determined by using dimethyl sulfoxide as a solvent, using tetramethylsilane as an internal standard and measuring the ¹H-NMR spectrum of the PVA resin completely saponified. Specifically, it can be calculated from peak areas derived from a proton of a hydroxyl group, a methine proton, a main chain methylene proton and a proton of a hydroxyl group connected with the main chain in the 1,2-diol configuration unit.

The saponification degree of the PVA resin used in the present invention is at least 99% by mol and nearly completely saponified articles to completely saponified articles are used. In particular, 99.3 to 99.9% by mol is preferable in practical use. The reason why the PVA resin having a high saponification degree is preferable is that the PVA resin with a high saponification degree is suitable for the precise control of embolization time that is the purpose of the present invention. The purpose of the present invention is to provide a temporary vascular embolic agent used for the preparation of the cardiac infarction model using animals; therefore it is required to embolize sites where blood flow rate led to cardiac is fast. The pearl like polyvinyl alcohol particles having not so high solubility to blood are rather suitable. Further, when the saponification degree is lowered, a saponification degree distribution between molecules is widened and as a result, great difference is generated in the time dissolving in blood between the PVA particles. The PVA particles can be dissolved in one shot by setting the high saponification degree and narrowly controlling the distribution of the saponification degree, finely adjusting the crystallinity of the PVA resin by thermal treatment by the content of the 1,2-diol configuration unit at side chains and controlling solubility in blood after the embolization of blood flow for a fixed time. As a result, the precise control of embolization time is possible.

The average diameter of the pearl like PVA particles is preferably 10 to 1200 μm. It is more preferably 30 to 1000 μm, further preferably 50 to 800 μm and particularly preferably 100 to 250 μm. When the average diameter is too large, passing property in a catheter is remarkably lowered depending on the kind of the catheter used, or passing tends to be impossible. On the other hand, when the average diameter of the pearl like PVA particles is too small, performance of embolizing the blood vessel is bad and embolization time is extremely shortened. Further, it is not preferable because an embolic agent is contaminated in the blood vessel such as, for example, circumflex other than the intended vessel. Further, the average diameter is a value measured at state in which a fixed amount of PVA is dispersed in isopropyl alcohol, unless otherwise noticed specifically in the present specification.

The average polymerization degree of the pearl like PVA particles is preferably 80 to 1500 when it is measured in accordance with JIS K6726, more preferably 90 to 1000 and further preferably 100 to 800. When the average polymerization degree is less than 80, it is not a polymerization degree at which stable production can be industrially carried out, and embolization time tends to be extremely shortened; therefore the preparation of an objective animal clinical condition model is difficult. When the average polymerization degree is at least 1500, the embolization time of particles in the blood vessel is very long and particles remains in vivo; therefore blood flow passes again and the preparation of the animal clinical condition model having necrosis site at the portion of cardiac muscle is difficult. Herein, the temporary embolic agent in the present invention means a temporary embolic agent by which the embolization time of the blood vessel is arbitrarily controlled in a range of about 30 min to 3 months.

The PVA resin used in the present invention has the configuration unit shown by the under-mentioned general formula (1A). R¹, R², R³, R⁴, R⁵ and R⁶ independently indicate a hydrogen atom or an organic group respectively and X indicates a single bond or a bond chain.

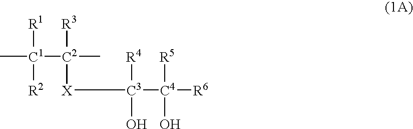

All of R¹ to R³ and R⁴ to R⁶ having the 1,2-diol configuration unit represented by the general formula (1A) are preferably hydrogen atoms. Provided that it may be substituted with an organic group so far as it is an amount not greatly damaging the property of the PVA resin. The organic group in such case is not specifically limited, but it is preferably alkyl groups having 1 to 4 carbon atoms such as, for example, a methyl group, an ethyl group, an n-propyl group, an isopropyl group, an n-butyl group, an isobutyl group and a tert-butyl group, and the alkyl group has further substituents such as a halogen group, a hydroxyl group, an ester group, a carboxyl group and a sulfone group, if necessary.

Further, X in the 1,2-diol configuration unit represented by the general formula (1A) is typically a single bond and the single bond is most preferable from the view points of thermal stability and stability under high temperature atmosphere or under oxidation condition. Herein, X of a single bond means that C² and C³ are singly bonded directly in the general formula (1). However, it may be a bond chain so far as the effect of the present invention is not obstructed. The bond chain is not specifically limited, but for example, hydrocarbons such as alkylene, alkenyl, phenylene and naphthylene are preferable and the hydrocarbon may be substituted with halogen such as fluorine, chlorine and bromine if necessary. Further, as the bond chain, there are mentioned —O—, —(CH₂O)ₘ—, —(OCH₂)ₘ—, —(CH₂O)ₘCH₂—, —CO—, —COCO—, —CO(CH₂)ₘCO—, —CO(C₆H₄)CO—, —S—, —CS—, —SO—, —SO₂—, —NR—, —CONR—, —NRCO—, —CSNR—, —NRCS—, —NRNR—, —HPO₄—, —Si(OR)₂—, —OSi(OR)₂—, —OSi(OR)₂O—, —Ti(OR)₂—, —OTi(OR)₂—, —OTi(OR)₂O—, —Al(OR)—, —OAl(OR)— and —OAl(OR)O— (provided that R's are independently an arbitrary group respectively, a hydrogen atom and an alkyl group are preferable and m is a natural number). Among these bond chains, —CH₂OCH₂— or an alkyl group having at most 6 carbon atoms, specifically a methylene group is preferable.

The production process of the PVA resin used in the present invention is not specifically limited, but there are preferable (i) a method of saponifying the copolymer of a vinyl ester monomer with a compound indicated the under-mentioned general formula (2), (ii) a method of saponifying and decarbonylating the copolymer of a vinyl ester monomer with a compound indicated the under-mentioned general formula (3), or (iii) a method of saponifying the copolymer of a vinyl ester monomer with a compound indicated the under-mentioned general formula (4) and carrying out the deketanolation of its saponified product.

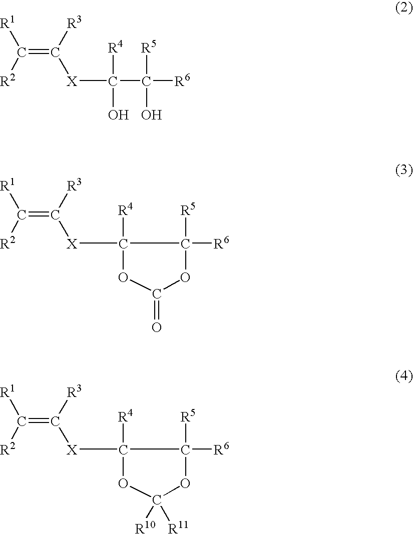

Herein, X, R¹, R², R³, R⁴, R⁵ and R⁶ in the above-mentioned general formulae (2), (3) and (4) are same as the general formula (1) and independently indicate a hydrogen atom or an organic group respectively, and X indicates a single bond or a bond chain. R⁷ and R⁸ are independently a hydrogen atom or R⁹—CO— (in the formula, R⁹ is an alkyl group) respectively. Further, R⁷ and R⁸ independently indicate a hydrogen atom or an organic group respectively.

As the production process of (i), (ii) and (iii), a production process described in Japanese Unexamined Patent Publication No. 2006-95825 can be adopted.

When the PVA resin is produced using the production process (i), 3,4-diacyloxy-1-butene in which R¹ to R⁶ is hydrogen, X is a single bond and either of R⁷ to R⁵ is R⁹—CO— is preferable as the compound indicated by the general formula (2) from the viewpoint of being superior in copolymerization reactivity and industrial processability. Among these, 3,4-diacetoxy-1-butene in which R⁹ is a methyl group is preferable in particular.

Further, when vinyl acetate is copolymerized with 3,4-diacetoxy-1-butene using vinyl acetate as the vinyl ester monomer, the reactivity ratio of respective monomers is r (vinyl acetate)=0.710 and r (3,4-diacetoxy-1-butene)=0.701. On the other hand, the reactivity ratio of vinyl ethylene carbonate that is the compound represented by the general formula (3) used in the production process (ii) is r (vinyl acetate)=0.85 and r (vinyl ethylene carbonate)=5.4. It is indicated from the comparison of the above-mentioned reactivity ratios that 3,4-diacetoxy-1-butene is superior in copolymerization reactivity with vinyl acetate.

Further, the chain transfer constant (Cx) of 3,4-diacetoxy-1-butene is Cx (3,4-diacetoxy-1-butene)=0.003 (65° C.). On the other hand, the chain transfer constant Cx (vinyl ethylene carbonate) of vinyl ethylene carbonate used in the production process (ii)=0.005 (65° C.) and the chain transfer constant Cx (2,2-dimethyl-4-vinyl-1,3-dioxolane) of 2,2-dimethyl-4-vinyl-1,3-dioxolane that is one of the compound represented by the general formula (4) used in the production process (iii)=0.023 (65° C.). It can be said from the comparison of these the chain transfer constants that since Cx of 3,4-diacetoxy-1-butene is low at 0.003, chain transfer does not occur exclusively, the polymerization degree tends to be not high and the lowering of polymerization speed is low.

Further, in the case of 3,4-diacetoxy-1-butene, a byproduct generated at saponifying a copolymer with a vinyl ester monomer is the same as a compound prepared as a byproduct at saponification, from configuration unit derived from vinyl acetate that is often used as the vinyl ester monomer. Consequently, a specific equipment and step are not required to be newly set for post treatment and solvent collection system and it is also industrially great advantage that conventional facilities used for vinyl acetate can be utilized.

Further, 3,4-diacetoxy-1-butene is commercially available as a product produced by a synthesis route described in, for example, International Unexamined Patent Publication No. 00/24702 in which 1,3-butadien is a starting substance and as a product that is produced using an epoxy butane derivative as an intermediate by technology described in U.S. Pat. No. 5,623,086 and U.S. Pat. No. 6,072,079. Further, it is available as a reagent from Across Co., Ltd. Further, it is available as a byproduct in the production step of 1,4-butanediol. It can be also utilized by purifying 3,4-diacetoxy-1-butene. Further, it can be also used by converting 1,4-diacetoxy-1-butene being an intermediate product in the production step of 1,4-butanediol to 3,4-diacetoxy-1-butene by known isomerization reaction using a metal catalyst such as palladium chloride. Further, it can be also produced in accordance with the production process of organic diester described in Republication No. 00/24702.

Further, when the PVA resin obtained by the production process (ii) or the production process (iii) is low in a saponification degree and inadequate in decarbonylation or deacetalation, a carbonate ring or an acetal ring remains occasionally at a side chain and as a result, the dissolution property of the pearl like PVA particles in blood is occasionally inhibited. It is also most preferable from these points that the PVA resin of the present invention is produced by the production process (i).

Other vinyl ester monomer composing the copolymer includes vinyl formate, vinyl acetate, vinyl isobutyrate, vinyl pyvarate, vinyl caprylate, vinyl laurylate, vinyl stearate, vinyl benzoate and vinyl versatate. Among these, vinyl acetate is preferable from the viewpoint of economics.

Further, as a copolymerization component, there can be also used α-olefins such as ethylene and propylene; α-olefins containing a hydroxyl group such as 3-buten-1-ol, 4-penten-1-ol and 5-hexen-1,2-diol; unsaturated carboxylic acids such as itaconic acid, maleic acid and acrylic acid; or salts thereof, or mono or dialkyl ester thereof, nitriles such as acrylonitrile; amides such as methacrylamide and diacetone acrylamide; and olefin sulfonates such as ethylene sulfonic acid, allyl sulfonic acid, methallyl sulfonic acid and 2-acrylamide-2-methyl sulfonic acid (AMPS) or salts thereof, other than the above-mentioned vinyl ester monomers and compounds indicated by the general formulae (2), (3) and (4), so far as they do not greatly affect the physical properties of the resin.

The pearl like PVA particles of the present invention can be produced according to a method of adding dropwise PVA aqueous solution with high concentration from a vibrating nozzle into a solution not dissolving PVA, for example, paraffin organic solvent and the cool aqueous solution of inorganic salts such as common salt, for example, using an equipment producing particles with small diameters manufactured by BRACE Inc. and according to the production process of granular polyvinyl alcohol described in Japanese Unexamined Patent Publication No. 2007-37989. Specifically, they can be obtained by granularly dispersing polyvinyl ester solution (b) in which alcohol or alcohol with methyl acetate (a) is solvent, into high viscous liquid (c) that is not substantially compatible with either of the polyvinyl ester, the saponified article of the ester and the component (a) and saponifying it in the presence of a saponification catalyst.

As the method of dispersing the polyvinyl ester solution (b) into the high viscous liquid (c), both are usually mixed and stirred, but in particular, more homogeneous dispersion condition is obtained by adding dropwise the polyvinyl ester solution (b) from a vibrating nozzle into the high viscous liquid (c) and the pearl like PVA particles having sharper particle size distribution and higher sphericity are obtained.

As the above-mentioned alcohol or alcohol and alcohol in the methyl acetate (a), lower aliphatic alcohols such as methanol, ethanol, isopropyl alcohol and propyl alcohol can be used. These alcohols can be used alone or by mixing at least 2 kinds at an arbitrary proportion. Among these, methanol, ethanol and isopropyl alcohol are preferably used from the viewpoints of controlling particle diameter at saponification reaction and of obtaining practical saponification speed. When alcohol and methyl acetate are used in combination, the proportion of alcohol/methyl acetate is preferably at least 0.5 by weight ratio from the viewpoint of saponification reaction efficiency of the polyvinyl ester and more preferably at least 1.5. Further, various organic solvents having further lower polarity than methyl acetate can be used in combination.

The content of polyvinyl ester in the polyvinyl ester solution (b) is not specifically limited but is preferably 10 to 80% by weight of the total solution. The polyvinyl ester solution (b) may include 0.05 to 10 parts by weight of water based on the polyvinyl ester. The distribution of trace residual acetic acid group of the saponified article is more random by the presence of a small amount of water and the small amount of water can carry out a role of controlling the saponification degree.

The high viscous liquid (c) that is not substantially compatible with either of the polyvinyl ester to be used, the saponified article of the ester, alcohol or alcohol with methyl acetate (a) and has higher viscosity than the polyvinyl ester solution (b) is preferable. For example, aliphatic saturated hydrocarbons such as liquid paraffin and kerosene, aromatic hydrocarbons and alicyclic hydrocarbons are mentioned. These can be used alone or at least 2 kinds can be used by mixing. Among these, liquid paraffin is preferable because the polyvinyl ester solution can be homogeneously dispersed.

The viscosity of the high viscous liquid (c) is not specifically limited so far as it is higher than the viscosity of the polyvinyl ester solution (b).

The use proportion of the polyvinyl ester solution (b) and the high viscous liquid (c) is preferably 2/8 to 6/4 by weight ratio and more preferably 4/6 to 5/5. When the use proportion of the polyvinyl ester solution (b) is less than 20% by weight, it is not preferable because production efficiency is lowered. When the use proportion of the polyvinyl ester solution (b) exceeds 60% by weight, dispersibility is bad, the aggregate of multi particles is easily firmed and the average diameter of the pearl like PVA particles tends to be large.

As the saponification catalyst, usual alkaline catalyst used for preparing the PVA resin by saponifying the polyvinyl ester can be used. The amount of the saponification catalyst used is suitably determined depending on the concentration of the polyvinyl ester and the objective saponification degree, but is preferably a proportion of 0.1 to 30 mmol based on vinyl acetate unit (1 mol) in the polyvinyl ester and that of 2 to 17 mmol.

The reaction temperature of the saponification reaction is preferably 20° C. to 60° C. When the reaction temperature is at most 20° C., reaction speed is lessened and reaction efficiency is lowered. When it exceeds 60° C., it is not preferable for safety because it is at least the boiling point of solvent.

The polyvinyl ester solution (b) with the high saponification degree of the present invention is preferably produced by saponification reaction at two stages because of safety purpose that toxicity to animals and human body by the property of the pearl like PVA particles to be obtained and liquid paraffin taken into the inside of the pearl like PVA particles and bad influence to the preparation of the infarction model are reduced. After the saponification reaction is carried out at primary saponification until the saponification degree is 75 to 90% by mol, particles are separated from reaction slurry by a solid-liquid separation equipment such as a centrifugal separation equipment and by filtration by Advantech filter paper No. 2 or No. 63 in laboratory, rinsing is carried out with suitable solvent or mix solvent such as methanol, methyl acetate, ethyl acetate and a mixture of methyl acetate with methanol if necessary and primary saponification particles are obtained. Successively, the primary saponification particles obtained are dispersed in alcohol solvents such as methanol and ethanol to carry out final saponification reaction. When the high saponification degree of at least 99% by mol demanded for the present invention was able to be attained, the reaction is terminated and the pearl like PVA particles (secondary saponification particles) of the present invention are obtained by a method similar as the collection of particles in the primary saponification. Then, rinsing is carried out with saline if necessary.

As the sterilization method of the pearl like PVA particles, there are used electron beam, ultraviolet rays, X ray, γ ray, ethylene oxide gas sterilization, pressured steam sterilization, a method of immersing in Hibiten solution (chlorohexidine gluconate solution) and a method of rinsing with normal saline solution for sterilization.

The average particle diameter of the PVA resin can be carried out by physically sieving the pearl like PVA particles obtained in the fore-mentioned production process with standard metallic mesh if necessary to carry out the adjustment of the pearl like PVA particles with arbitrary particle diameter. Further, in order to lessen the average particle diameter to a desired level, stirring speed at saponification reaction at production in accordance with the production process of granular polyvinyl alcohol described in Japanese Unexamined Patent Publication No. 56-120707 is enhanced, the viscosity of the high viscous liquid (c) such as liquid paraffin is more highly set than the viscosity of the polyvinyl ester solution (b) and the ratio of the high viscous liquid (c) to the polyvinyl ester solution (b) is controlled; therefore their particle diameter can be also controlled.

For example, when the particle diameter is set at a range of 105 to 177 μm, those having particle diameter sieved by 145 mesh (105 μm) on and 80 mesh (177 μm) pass are used. Further, when it is set at a range of 177 to 297 μm, those having particle diameter sieved by 80 mesh (177 μm) on and 48 mesh (297 μm) pass are used, and when it is set at 297 to 500 μm, those having particle diameter sieved by 48 mesh (297 μm) on and 32 mesh (500 μm) pass are used.

Further, a specific method of obtaining the pearl like PVA particles with a desired average particle diameter by the saponification reaction described above is illustrated as follow. For example, in order to obtain the pearl like PVA particles with an average particle diameter of about 150 μm, in the case of the polyvinyl ester having an average polymerization degree of 500, the concentration of the methanol solution (b) is set as 40% by weight, and in the case of the polyvinyl ester having an average polymerization degree of 150 to 200, the concentration of the methanol solution (b) is set as 50% by weight and the proportion of the solution (b) to the high viscous liquid (c) such as liquid paraffin is set as 50/50 by weight ratio to carry out the saponification reaction. Further, in order to make an average particle diameter of about 50 μm, the viscosity of the polyvinyl ester solution (b) may be set at a higher value than that of the high viscous liquid (c) and for example, the resin concentration of the polyvinyl ester solution having an average polymerization degree of 500 is set as 50% by weight.

The temporary vascular embolic agent (i) can be prepared by dispersing the pearl like PVA particles obtained by the above-mentioned method in a contrast agent.

As the contrast agent, either of an ionic contrast agent and a nonionic contrast agent can be used. Specifically, it includes Topamiron (manufactured by Bayer Schering Pharma AG), Oypalomin (manufactured by Fuji Pharma Co., Ltd.), Hexabrix (manufactured by Terumo Corporation), Omnipaque (manufactured by Daiichi-Sankyo Co. Ltd.), Urografin (manufactured by Bayer Schering Pharma AG) and Tomeron (manufactured by Eisai Co., Ltd.).

The pearl like PVA particles are preferably used at a proportion of at most 20% by weight for the contrast agent from the viewpoint that catheter passing property is required to be secured. In this case, after dispersing the pearl like PVA particles in the contrast agent and the dispersion is left alone for 5 to 15 min, they are preferably used as the temporary vascular embolic agent. Time until disobliteration after embolization can be controlled by the polymerization degree of the PVA resin, the saponification degree, the content of 1,2-diol configuration unit and release time in the contrast agent. Embolization time can be elongated by enhancing the polymerization degree and saponification degree of the PVA resin and by reducing the content of the 1,2-diol configuration unit. Further, when preliminary release time in the contrast agent is elongated, embolization time is shortened. Preliminary release time in the contrast agent affects greatly the control of embolization time. When the release time is less than 5 min, the swelling of the pearl like PVA particles by the contrast agent is inadequate and time until the re-dissolution of the pearl like PVA particles after embolization in the blood vessel tends to be elongated. On the other hand, when it exceeds 15 min, the pearl like PVA particles are inversely swollen by the contrast agent excessively; therefore the pearl like PVA particles become patching powder condition and are easily adhered on the interior wall of a catheter; therefore the passing property of a catheter is lowered and the workability of embolization remedy tends to be remarkably lowered.

Further, the temporary vascular embolic agent (ii) can be prepared by dissolving the pearl like PVA particles of the present invention in the contrast agent. Specifically, at most 20% by weight of the pearl like PVA particles are added to 100 parts by weight of the contrast agent, the mixture is heated at about 50° C. to 70° C. and it is dissolved for about 30 minutes to 2 hours to obtain the paste temporary vascular embolic agent.

Furthermore, the temporary vascular embolic agent (iii) can be prepared by dispersing the pearl like PVA particles of the present invention in the fore-mentioned paste temporary vascular embolic agent. The embolic agent can control embolization time by changing the mixing weight ratio of the PVA resin (A) dissolved in the paste temporary vascular embolic agent to the pearl like PVA particles (B) being in dispersion condition. When the proportion of the PVA resin (A) dissolved is too small in the mixing, embolization time in the blood vessel is occasionally too long depending on the diameter of the blood vessel at embolization in the blood vessel. When it is too large, embolization time is extremely short easily (for example, about 15 min) and objective embolization time is not occasionally obtained.

Therapeutic ingredient may be added in the temporary vascular embolic agent of the present invention. The therapeutic ingredient can be compounded in the temporary vascular embolic agent by a method of occluding and supporting it on the pearl like PVA particles at mixing the contrast agent with the pearl like PVA particles and in solvent dissolving the therapeutic ingredient. The therapeutic ingredient includes chemical therapeutic agents such as anticancer drugs such as SMANCS and cyclophosphamide, steroid hormone drugs, hepatic disease drugs, diabetic medicine, antioxidants, peptide drugs, molecular target remedy for cancer and antibiotic drugs and thrombus forming inhibitors such as heparin. Further, there are mentioned basic fibrocyte growth factor (bFGF), platelet-derived growth factor (PDGF), transforming growth factor β1 (TGF-β1) and vascular endothelial growth factor (VEGF) that are cell growth factor.

The catheter used at embolizing the temporary vascular embolic agent of the present invention in blood is not specifically limited and a catheter, MASS TRANSIT manufactured by CORDIS Co. and a catheter, Progreat manufactured by Terumo Corporation can be suitably selected.

### EXAMPLES

The pearl like PVA particles and the production process of the particles and the temporary vascular embolic agent of the present invention are more specifically illustrated below based on Examples, but the present invention is not limited to only Examples. Further, “parts” means “parts by weight” and “%” means “% by weight” unless otherwise noticed.

### Example 1

(Production of pearl like PVA particles A; an average particle diameter of 150 μm, a saponification degree of 99.6% by mol, pearl like saponified article, an average polymerization degree of 500 and a denaturation rate of 0.3% by mol)

Into a reaction vessel equipped with a reflux cooler, a dropping funnel and a stirrer, 900 g of vinyl acetate, 1440 g of methanol and 5.4 g of 3,4-diacetoxy-1-butene (hereinafter, called as 3,4DAB) were charged, 0.2% by mol (for vinyl acetate fed) of azoisobutyronitrile was charged therein, and temperature was raised under nitrogen atmosphere while stirring, to start polymerization. In this case, the denaturation rate of vinyl acetate by 3,4DAB was about 0.3% by mol.

When the polymerization rate of vinyl acetate was 92%, 20 ppm of m-dinitrobenzene was added to terminate polymerization. Successively, unreacted vinyl acetate monomer was removed out of the system by a method of blowing methanol vapor to obtain the methanol solution of (vinyl acetate-3,4DAB) copolymer.

Methanol was added to the methanol solution of (vinyl acetate-3,4DAB) copolymer obtained and resin content was adjusted at 40%. 100 Parts of the solution was fed in a reaction vessel with a stirrer, temperature was kept at 30° C. and 2% methanol solution converted to Na content of NaOH was added as saponification reaction catalyst at a proportion of 3 mmol for the vinyl acetate unit of polyvinyl acetate while stirring. Successively, when 100 parts of liquid paraffin was added thereto and stirring speed was adjusted at 300 rpm, polyvinyl acetate was dispersed in liquid paraffin in spherical shape. Reaction was carried out by keeping temperature at 30° C., the reaction was terminated after the lapse of 60 min, and pearl like PVA particles were separated by carrying out solid-liquid separation by a centrifugal separation equipment. The particles were rinsed by extraction method using ethyl acetate solution at temperature of 50° C. and then, were dried at temperature of 80° C. for 24 hrs using a vacuum drier.

100 Parts of the pearl like PVA particles obtained (primary saponified particles) were dispersed again in 500 parts of methanol solution, 20 parts of saponification catalyst (2% methanol solution converted to Na content of NaOH) was added thereto and secondary saponification was carried out at temperature of 50° C. for 2 hrs. Then, the pearl like PVA particles were separated again by the centrifugal separation equipment, rinsed by extraction method using ethyl acetate solution at temperature of 50° C. and dried at temperature of 80° C. for 24 hrs using the vacuum drier to prepare the pearl like PVA particles A.

When the pearl like PVA particles A were measured with ¹H-NMR using DMSO-d6 as solvent and setting tetramethylsilane as internal standard, the content of the side chain of 1,2-diol configuration was 0.3% by mol.

### (Average Polymerization Degree)

It was measured in accordance with JIS K6726 and as a result, the average polymerization degree of the pearl like PVA particles was 500.

### (Saponification Degree)

The saponification degree of the pearl like PVA particles was also measured in accordance with JIS K6726 using the consumption amount required for the hydrolysis of residual vinyl acetate and 3,4DAB. The saponification degree of the pearl like PVA particles measured was 99.6% by mol.

### (Average Particle Diameter)

The average particle diameter of the pearl like PVA particles was carried out by dispersing PVA particles (100 parts by weight) in isopropyl alcohol (100 parts by weight) and measuring average cord length (μm) with LASENTEC M100 (inline type particle monitoring system manufactured by LASENTEC Co.), to obtain average particle diameter. Specifically, a range of 0.8 to 1200 μm was divided into 38 channels cord length and the number of respective particles was counted to determine it by the formula (2) below.

Average cord length=Σ(Yi×Mi)/Σyi (2)

(Wherein Yi is the count number of particles at monitoring with the LASENTEC M100 and Mi is the cord length of respective channels.)

The average particle diameter of the pearl like PVA particles measured by the above-mentioned method was 150 μm.

### Comparative Example 1

PVA particles V were prepared according to the method of Example 1 except that unmodified PVA with a saponification degree of 99.3% by mol and an average polymerization degree of 300 was used as PVA. In the case of Comparative Example 1, the denaturation rate of vinyl acetate by 3,4DAB is 0% by mol.

### Example 2

After charging a predetermined amount of the pearl like PVA particles A of Example 1 into an eggplant-shaped flask, the eggplant-shaped flask was installed on a rotary evaporator. After air in the eggplant-shaped flask was replaced with nitrogen, the eggplant-shaped flask was immersed in an oil bath at predetermined temperature and thermal treatment was carried out by rotating it for a fixed time. Example 2 changed thermal treatment condition by changing the temperature of the oil bath and rotational time. Conditions by every Example are shown in Table 1.

### Comparative Examples 2 and 3

The PVA particles V of Comparative Example 1 was thermally treated by a method similar as Example 2. Comparative Examples 2 to 3 changed thermal treatment condition by changing the temperature of the oil bath and rotational time. Conditions by every Comparative Example are shown in Table 1.

### Example 3

(Production of pearl like PVA particles B; an average particle diameter of 150 μm, a saponification degree of 99.7% by mol, pearl like saponified article, an average polymerization degree of 470 and a denaturation rate of 0.5% by mol)

Into a reaction vessel equipped with a reflux cooler, a dropping funnel and a stirrer, 900 g of vinyl acetate, 1440 g of methanol and 9 g of 3,4-diacetoxy-1-butene (hereinafter, called as 3,4DAB) were charged, 0.3% by mol (for vinyl acetate fed) of azoisobutyronitrile was charged therein, and temperature was raised under nitrogen atmosphere while stirring, to start polymerization. In this case, the denaturation rate of vinyl acetate by 3,4DAB was about 0.5% by mol.

When the polymerization rate of vinyl acetate was 98%, 20 ppm of m-dinitrobenzene was added to terminate polymerization. Successively, unreacted vinyl acetate monomer was removed out of the system by a method of blowing methanol vapor to obtain the methanol solution of (vinyl acetate-3,4DAB) copolymer.

Methanol was added to the methanol solution of (vinyl acetate-3,4DAB) copolymer obtained and resin content was adjusted at 40%. 100 Parts of the solution was fed in a reaction vessel with a stirrer, temperature was kept at 30° C. and 2% methanol solution converted to Na content of NaOH was added as saponification reaction catalyst at a proportion of 3.2 mmol for the vinyl acetate unit of polyvinyl acetate while stirring. Successively, when 100 parts of liquid paraffin was added thereto and stirring speed was adjusted at 300 rpm, polyvinyl acetate was dispersed in liquid paraffin in spherical shape. Reaction was carried out by keeping temperature at 30° C., the reaction was terminated after the lapse of 60 min and pearl like PVA particles were separated by carrying out solid-liquid separation by a centrifugal separation equipment. The particles were rinsed by extraction method using ethyl acetate solution at temperature of 50° C. and then, were dried at temperature of 80° C. for 24 hrs using a vacuum drier.

100 Parts of the pearl like PVA particles obtained (primary saponified particles) were dispersed again in 500 parts of methanol solution, 20 parts of saponification catalyst (2% methanol solution converted to Na content of NaOH) was added thereto and secondary saponification was carried out at temperature of 50° C. for 2 hrs. Then, the pearl like PVA particles were separated again by the centrifugal separation equipment, rinsed by extraction method using ethyl acetate solution at temperature of 50° C. and dried at temperature of 80° C. for 24 hrs using the vacuum drier to prepare the pearl like PVA particles A.

When the pearl like PVA particles B were measured with ¹H-NMR using DMSO-d6 as solvent and setting tetramethylsilane as internal standard, the content of the side chain of 1,2-diol configuration was 0.5% by mol.

### Examples 4 and 5

After charging a predetermined amount of the pearl like PVA particles B of Example 3 into an eggplant-shaped flask, the eggplant-shaped flask was installed on a rotary evaporator. After air in the eggplant-shaped flask was replaced with nitrogen, the eggplant-shaped flask was immersed in an oil bath at predetermined temperature and thermal treatment was carried out by rotating it for a fixed time. Examples 4 and 5 changed thermal treatment condition by changing the temperature of the oil bath and rotational time. Conditions by every Example are shown in Table 1.

### Example 6

(Production of pearl like PVA particles C; an average particle diameter of 150 μm, a saponification degree of 99.7% by mol, pearl like saponified article, an average polymerization degree of 480 and a denaturation Rate of 1.5% by mol)

Into a reaction vessel equipped with a reflux cooler, a dropping funnel and a stirrer, 900 g of vinyl acetate, 1440 g of methanol and 27 g of 3,4-diacetoxy-1-butene (hereinafter, called as 3,4DAB) were charged, 0.3% by mol (for vinyl acetate fed) of azoisobutyronitrile was charged therein, and temperature was raised under nitrogen atmosphere while stirring, to start polymerization. In this case, the denaturation rate of vinyl acetate by 3,4DAB was about 1.5% by mol.

When the polymerization rate of vinyl acetate was 96.5%, 20 ppm of m-dinitrobenzene was added to terminate polymerization. Successively, unreacted vinyl acetate monomer was removed out of the system by a method of blowing methanol vapor to obtain the methanol solution of (vinyl acetate-3,4DAB) copolymer.

Methanol was added to the methanol solution of (vinyl acetate-3,4DAB) copolymer obtained and resin content was adjusted at 40%. 100 Parts of the solution was fed in a reaction vessel with a stirrer, temperature was kept at 30° C. and 2% methanol solution converted to Na content of NaOH was added as saponification reaction catalyst at a proportion of 3.5 mmol for the vinyl acetate unit of polyvinyl acetate while stirring. Successively, when 100 parts of liquid paraffin was added thereto and stirring speed was adjusted at 300 rpm, polyvinyl acetate was dispersed in liquid paraffin in spherical shape. Reaction was carried out by keeping temperature at 30° C., the reaction was terminated after the lapse of 60 min and pearl like PVA particles were separated by carrying out solid-liquid separation by a centrifugal separation equipment. The particles were rinsed by extraction method using ethyl acetate solution at temperature of 50° C. and then, were dried at temperature of 80° C. for 24 hrs using a vacuum drier.

100 Parts of the pearl like PVA particles obtained (primary saponified particles) were dispersed again in 500 parts of methanol solution, 22 parts of saponification catalyst (2% methanol solution converted to Na content of NaOH) was added thereto and secondary saponification was carried out at temperature of 50° C. for 2 hrs. Then, the pearl like PVA particles were separated again by the centrifugal separation equipment, rinsed by extraction method using ethyl acetate solution at temperature of 50° C. and dried at temperature of 80° C. for 24 hrs using the vacuum drier to obtain the pearl like PVA particles C.

When the pearl like PVA particles C were measured with ¹H-NMR using DMSO-d6 as solvent and setting tetramethylsilane as internal standard, the content of the side chain of 1,2-diol configuration was 1.5% by mol.

### Examples 7 and 8

After charging a predetermined amount of the pearl like PVA particles C of Example 6 into an eggplant-shaped flask, the eggplant-shaped flask was installed on a rotary evaporator. After air in the eggplant-shaped flask was replaced with nitrogen, the eggplant-shaped flask was immersed in an oil bath at predetermined temperature and thermal treatment was carried out by rotating it for a fixed time. Examples 7 and 8 changed thermal treatment condition by changing the temperature of the oil bath and rotational time. Conditions by every Example are shown in Table 1.

### Comparative Example 4

PVA particles W were prepared according to the method of Example 1 except that unmodified PVA with a saponification degree of 99.7% by mol and an average polymerization degree of 450 was used as PVA. In the case of Comparative Example 4, the denaturation rate of vinyl acetate by 3,4DAB is 3% by mol.

### Comparative Examples 5 and 6

The PVA particles W of Comparative Example 4 was thermally treated by a method similar as Example 2. Comparative Examples 5 and 6 changed thermal treatment condition by changing the temperature of the oil bath and rotational time. Conditions by every Comparative Example are shown in Table 1.

### (Evaluation of Solubility)

Into a 100 ml beaker, 70 g of water was charged, 3 g of the pearl like PVA particles sieved to 100 to 212 μm were charged thereto and the mixture was stirred for 2 min. Then, the beaker was immersed in a water vessel at 37° C. and the particle number of the PVA particles using LASENTEC M100F (manufactured by LASENTEC Inc., an uptake time of 24.75 sec). The ratio of particle number after 3 hrs to particle number just after measurement start was referred to as residual rate and the residual rate was adopted as the evaluation index of solubility. The result measuring the residual rate for the PVA particles of Examples 1 to 8 and Comparative Examples 1 to 6 is shown in Table 1.

### (Evaluation of Passing Property of Catheter)

On petri dishes with a diameter of 5 cm, 2 g of the pearl like PVA particles of Example 1 to 8 and Comparative Examples 1 to 6 were put and 8 g of a contrast agent (Oypalomin 300) was charged thereto. After stirring with a drug spatula for 1 min, the mixtures were soaked up with 2 cc syringes, they were connected with catheters (Micro catheter 2.3 Fr) and pistons were pushed in to push PVA/contrast agent solution. Evaluation basis is as below. Result is shown in Table 1.

### (Evaluation Basis)

o: It could be pushed out without resistance.
Δ: Although resistance was felt, it could be pushed out.
x: It could not be pushed out because of resistance.

Into a 100 ml beaker, 70 g of water was charged, 3 g of the pearl like PVA particles sieved to 100 to 212 μm were charged thereto and the mixture was stirred for 2 min. Then, the beaker was immersed in a water vessel at 37° C. and the particle number of the PVA particles using LASENTEC M100F (manufactured by LASENTEC Inc., an uptake time of 24.75 sec). The ratio of particle number after 3 hrs to particle number just after measurement start was referred to as residual rate and the residual rate was adopted as the evaluation index of solubility. The result measuring the residual rate for the PVA particles of Examples 1 to 8 and Comparative Examples 1 to 6 is shown in Table 1.

It is grasped from the result that when the pearl like PVA particles of the present invention are filled in the blood vessel as a temporary vascular embolic agent, the dissolution speed of their particle groups is not too fast and suitable and since the dissolution speed can be adjusted by the content of 1,2-diol configuration unit and thermal treatment condition, it is considered to be useful because a temporary vascular embolic agent according to purposes is easily obtained. Further, a catheter can be passed without resistance.

On the other hand, in the case of the PVA without denaturation of Comparative Examples, the dissolution speed is too fast when thermal treatment is not carried out and the dissolution speed is hardly controlled depending on the level of thermal treatment; therefore it is difficult to obtain PVA particles having appropriate dissolution speed. Further, it is difficult or impossible to pass a catheter.

TABLE 1

| | | | Com. | Com. | Com. | | | | | | | Com. | Com. | Com. |
|---|---|---|---|---|---|---|---|---|---|---|---|---|---|---|
| | Ex. 1 | Ex. 2 | Ex. 1 | Ex. 2 | Ex. 3 | Ex. 3 | Ex. 4 | Ex. 5 | Ex. 6 | Ex. 7 | Ex. 8 | Ex. 4 | Ex. 5 | Ex. 6 |
| denaturation | 0.3 | 0.3 | 0 | 0 | 0 | 0.5 | 0.5 | 0.5 | 1.5 | 1.5 | 1.5 | 3 | 3 | 3 |
| rate | | | | | | | | | | | | | | |
| (% by mol) | | | | | | | | | | | | | | |
| saponification | 99.6 | 99.6 | 99.3 | 99.3 | 99.3 | 99.7 | 99.7 | 99.7 | 99.7 | 99.7 | 99.7 | 99.7 | 99.7 | 99.7 |
| degree | | | | | | | | | | | | | | |
| (% by mol) | | | | | | | | | | | | | | |
| polymerization | 500 | 500 | 300 | 300 | 300 | 470 | 470 | 470 | 480 | 480 | 480 | 450 | 450 | 450 |
| degree | | | | | | | | | | | | | | |
| average particle | 150 | 150 | 150 | 150 | 150 | 150 | 150 | 150 | 150 | 150 | 150 | 150 | 150 | 150 |
| diameter (μm) | | | | | | | | | | | | | | |
| thermal | — | 120 | — | 120 | 130 | — | 120 | 130 | — | 120 | 130 | — | 120 | 130 |
| treatment | | | | | | | | | | | | | | |
| temperature | | | | | | | | | | | | | | |
| (° C.) | | | | | | | | | | | | | | |
| thermal | 0 | 30 | 0 | 0 | 30 | 0 | 30 | 30 | 0 | 30 | 30 | 0 | 30 | 30 |
| treatment | | | | | | | | | | | | | | |
| time (min) | | | | | | | | | | | | | | |
| evaluation of | | | | | | | | | | | | | | |
| solubility | | | | | | | | | | | | | | |
| initial numbers | 22387 | 24704 | 18060 | 25418 | 34468 | 22512 | 24192 | 27596 | 18817 | 19614 | 26091 | 17648 | 18639 | 25337 |
| of perticles | | | | | | | | | | | | | | |
| residual | 8425 | 14730 | 4941 | 6581 | 34275 | 7654 | 12580 | 23457 | 5645 | 8434 | 15655 | 1412 | 2609 | 6334 |
| numbers of | | | | | | | | | | | | | | |
| perticles | | | | | | | | | | | | | | |
| residual rate | 38 | 60 | 27 | 26 | 99 | 34 | 52 | 85 | 30 | 43 | 60 | 8 | 14 | 25 |
| (%) | | | | | | | | | | | | | | |
| evaluation of | ∘ | ∘ | Δ | Δ | ∘ | ∘ | ∘ | ∘ | ∘ | ∘ | ∘ | x | x | Δ |
| passing | | | | | | | | | | | | | | |
| property of | | | | | | | | | | | | | | |
| catheter | | | | | | | | | | | | | | |

1-5. (canceled)

6. A pearl like polyvinyl alcohol particle comprising a polyvinyl alcohol resin having 0.1 to 2% by mol of a 1,2-diol configuration unit indicated by the following general formula (1) and having a saponification degree of at least 99% by mol, 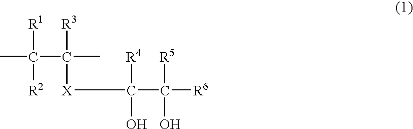
wherein R¹, R² and R³ independently indicate a hydrogen atom or an organic group respectively, X indicates a single bond or a bond chain, and R⁴, R⁵ and R⁶ independently indicate a hydrogen atom or an organic group respectively.

7. The particle of claim 6, wherein R¹, R², R³, R⁴, R⁵ and R⁶ in said general formula (1) are hydrogen atoms or alkyl groups having 1 to 4 carbon atoms.

8. The particle of claim 6, wherein R¹, R², R³, R⁴, R⁵ and R⁶ in said general formula (1) are hydrogen atoms and X is a single bond.

9. A process for producing a pearl like polyvinyl alcohol particle comprising a polyvinyl alcohol resin having 0.1 to 2% by mol of a 1,2-diol configuration unit indicated by the following general formula (1) and having a saponification degree of at least 99% by mol, 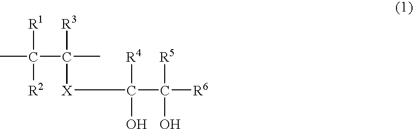
wherein R¹, R² and R³ independently indicate a hydrogen atom or an organic group respectively, X indicates a single bond or a bond chain, and R⁴, R⁵ and R⁶ independently indicate a hydrogen atom or an organic group respectively, and
wherein a polyvinyl ester copolymer solution obtained by copolymerizing a vinyl ester monomer with a monomer capable of being the 1,2-diol configuration unit indicated by the general formula (1) by saponification is saponified while being dispersed in a specific high viscous liquid.

10. The process of claim 9, wherein R¹, R², R³, R⁴, R⁵ and R⁶ in said general formula (1) are hydrogen atoms or alkyl groups having 1 to 4 carbon atoms.

11. The process of claim 9, wherein R¹, R², R³, R⁴, R⁵ and R⁶ in said general formula (1) are hydrogen atoms and X is a single bond.

12. A temporary vascular embolic agent comprising a pearl like polyvinyl alcohol particle comprising a polyvinyl alcohol resin having 0.1 to 2% by mol of a 1,2-diol configuration unit indicated by the following general formula (1) and having a saponification degree of at least 99% by mol, 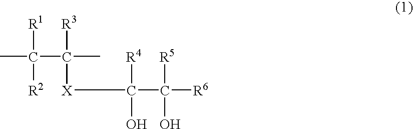
wherein R¹, R² and R³ independently indicate a hydrogen atom or an organic group respectively, X indicates a single bond or a bond chain, and R⁴, R⁵ and R⁶ independently indicate a hydrogen atom or an organic group respectively.

13. The agent of claim 12, wherein R¹, R², R³, R⁴, R⁵ and R⁶ in said general formula (1) are hydrogen atoms or alkyl groups having 1 to 4 carbon atoms.

14. The agent of claim 12, wherein R¹, R², R³, R⁴, R⁵ and R⁶ in said general formula (1) are hydrogen atoms and X is a single bond.
